(19) United States
(12) Patent Application Publication
(10) Pub. No.: US 2019/0195858 A1
(43) Pub. Date: Jun. 27, 2019

(54) Separation of Rare Cells and Genomic Analysis Thereof

(71) Applicant: Emory University, Atlanta, GA (US)

(72) Inventors: Adam Marcus, Decatur, GA (US); Jessica Konen, Houston, TX (US)

(21) Appl. No.: 16/099,431

(22) PCT Filed: May 5, 2017
(86) PCT No.: PCT/US17/31341

§ 371 Date: Nov. 6, 2018

Related U.S. Application Data
(60) Provisional application No. 62/332,631, filed on May 6, 2016.

(51) Int. Cl. G01N 33/50; C12Q 1/6886; G01N 33/574; G01N 21/64

(52) U.S. Cl. CPC G01N 33/5005; C12Q 1/6886; C12Q 2600/112; G01N 21/6428; G01N 33/574

(57) ABSTRACT

This disclosure relates to method of isolating rare cells, e.g. unique cancer cells. In certain embodiments, the disclosure contemplates methods of identifying unique cells that are in a sample that contains a group of cells that are replicating at unique locations and/or at different rates. In certain embodiments the uniquely isolated and/or identified cells are evaluated for genetic content and/or expression for diagnostic evaluations. In certain embodiments, the disclosure contemplates compositions comprising cells that are derived from methods disclosed herein.

### CROSS-REFERENCE TO RELATED APPLICATIONS

This application claims the benefit of U.S. Provisional Application No. 62/332,631 filed May 6, 2016. The entirety of this application is hereby incorporated by reference for all purposes.

### STATEMENT REGARDING FEDERALLY SPONSORED RESEARCH

This invention was made with government support under 5R21CA201744-02 awarded by the National Institutes of Health. The government has certain rights in the invention.

### BACKGROUND

Patients with metastatic disease often develop multidrug resistance and succumb to cancer. In lung cancer, two mutations have been discovered (EGFR mutations, ALK translocations) that can direct someone to pursue optimal chemotherapy treatments. However, directed chemotherapy is not always effective. Intratumour genetic heterogeneity (ITH) is thought to contribute to therapeutic failure. See Burrell et al., Mol Oncol. 2014, 8(6):1095-111. Rare cell sub-populations within the bulk of a tumor are often considered the drivers of cell proliferation, survival, and metastasis. Rare cells are thought to survive treatment and repopulate the tumor. Thus, there is a need to identify and characterize rare cells in tumors in order to improve treatment options.

Konen & Marcus report a technique to provide spatiotemporal genomic profiling of rare cancer cells. Cancer Res, 2015, 75(22 Suppl 1):Abstract nr A1-18.

Patterson, et al report photoactivatable GFP for selective photolabeling of proteins and cells. Science, 2002, 297(5588):1873-1877. Mellott et al. report fluorescent photo-conversion to label unique cells. Cell Mol Bioeng. 2015, 8(1):187-196. See also US Patent Application Publication 20120295798, 2011/0296538, Yaron et al., Biol Proced Online. 2014, 16:9; Wlodkowic et al., Anal Chem. 2009, 81(13):5517-23.

References cited herein are not an admission of prior art.

### SUMMARY

This disclosure relates to method of isolating rare cells, e.g. unique cancer cells. In certain embodiments, the disclosure contemplates methods of identifying unique cells that are in a sample that contains a group of cells that are replicating at unique locations and/or at different rates. In certain embodiments the uniquely isolated and/or identified cells are evaluated for genetic content and/or RNA expression for diagnostic evaluations. In certain embodiments, the disclosure contemplates compositions comprising cells that are derived from methods disclosed herein.

In certain embodiments, the disclosure relates to methods of selecting unique cells comprising: a) mixing a group of cells suspected of containing cancer cells with a fluorescent photo-convertible protein, dye, or a recombinant vector configured to expresses a fluorescent photo-convertible protein, wherein the fluorescent photo-convertible protein or dye is configured to change fluorescent emissions if exposed to a predetermined wavelength of electromagnetic radiation, under conditions such that the group of cells contain the fluorescent photo-convertible protein or dye providing fluorescent protein or dye containing cells; b) providing conditions such that the fluorescent protein or dye containing cells replicate providing fluorescent replicated cells; c) identifying a fluorescent replicated cell that is expressing a physical characteristic that is unique compared to the other fluorescent replicated cells providing a unique fluorescent replicated cell; d) exposing the unique fluorescent replicated cell to the predetermined wavelength of electromagnetic radiation and not exposing the predetermined wavelength to the other fluorescent replicated cells under conditions such that the unique fluorescent replicated cell changes fluorescent emissions providing a changed unique fluorescent replicated cell; or exposing the other fluorescent replicated cells to the predetermined wavelength of electromagnetic radiation and not exposing the predetermined wavelength to the unique fluorescent replicated cell under conditions such that the other fluorescent replicated cells change fluorescent emissions providing changed other fluorescent replicated cells; and e) separating the changed unique fluorescent replicated cell from the other fluorescent replicated cells or separating the unique fluorescent replicated cells from the changed other fluorescent replicated cells.

In certain embodiments, the method further comprises the step of replicating the changed unique fluorescent replicated cell or the unique fluorescent replicated cells.

In certain embodiments, the physical characteristic that is unique is a location of the fluorescent replicated cell that are outward from a central mass of fluorescent replicated cells.

In certain embodiments, the physical characteristic that is unique is a location of the fluorescent replicated cell in a shape that is distinct from the shape of a central mass of fluorescent replicated cells.

In certain embodiments, the physical characteristic that is unique is a location of the fluorescent replicated cell at the tip of an arm of fluorescent replicated cells. In certain embodiments, the cells are leader cells or follower cells.

In certain embodiments, a sample or a mixture of cells is a tumor, organ biopsy, blood cells, urine cells, skin cells, tongue cells, cheek cells, fecal cells, or vaginal cells.

In certain embodiments, a sample or mixture of cells are lung cells, brain cells, kidney cells, pancreatic cells, ovarian cells, prostate cells or tumors thereof.

In certain embodiments, the unique fluorescent replicated cell is a metastasized or invasive cancer cell.

In certain embodiments, the fluorescent protein is selected from a fluorescent photo-convertible protein, photoactivatable fluorescent protein (PAFP), PA-GFP, PS-CFP, PS-CFP2, Kaede, EosFP, monomeric mEosFP, KikGR, Dendra, Dendra2, a kindling fluorescent proteins (KFPs), Dronpa, rsFastLime, mTFP0.7.

In certain embodiments, the method further comprises extracting nucleic acids from the unique fluorescent replicated cell providing extracted nucleic acids.

In certain embodiments, the method further comprises sequencing the extracted nucleic acids providing a unique florescent replicated cell sequence.

In certain embodiments, the method further comprises comparing the unique florescent replicated cell sequence to a standard or normal sequence and identifying similarities or differences between unique florescent replicated cell sequence and the standard or normal sequence.

In certain embodiments, the method further comprises recording the similarities or differences on a computer readable medium.

In certain embodiments, the disclosure relates to compositions comprising cells made by the processes disclosed herein.

In certain embodiments, the composition is characterized by contain a majority of cells that express a mutant sequence. In certain embodiments, the majority is greater than 60%, 70%, 80%, 90%, 95%, 96%, 97%, 98%, or 99% of the cells in the composition.

### BRIEF DESCRIPTION OF THE FIGURES

FIG. 1 shows scheme of a method of this disclosure where any cell(s) in a spheroid, 2-D culture, or 3-D overlay of cells on a tissue, are photoconverted green to red, sorted, and subjected to mRNA or other genomic profiling.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In the claims appended hereto, the term “a” or “an” is intended to mean “one or more,” and the term “comprise” and variations thereof such as “comprises” and “comprising,” when preceding the recitation of a step or an element, are intended to mean that the addition of further steps or elements is optional and not excluded.

The term “fluorescence-activated cell sorting” or “FACS” refers to a method of sorting a mixture of cells into two or more areas, typically one cell at a time, based upon the fluorescent characteristics of each cell, a respectively applied electrical charge, and separation by movement through an electrostatic field. Typically, a vibrating mechanism causes a stream of cells to break into individual droplets. Just prior to droplet formation, cells in a fluid pass through an area for measuring fluorescence of the cell. An electrical charging mechanism is configured at the point where the stream breaks into droplets. Based on the fluorescence intensity measurement, a respective electrical charge is imposed on the droplet as it breaks from the stream. The charged droplets then move through an electrostatic deflection system that diverts droplets into areas based upon their relative charge. In some systems, the charge is applied directly to the stream, and the droplet breaking off retains charge of the same sign as the stream. The stream is then returned to neutral after the droplet breaks off. In other systems, a charge is provided on a conduit inducing an opposite charge on the droplet.

The term “recombinant vector encoding” a specified polypeptide refers to nucleic acid sequence which encodes a gene product wherein then entire sequence of the nucleic acid is not naturally occurring. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. are be placed in close proximity to the coding region of the gene if needed to permit proper initiation of transcription and/or correct processing of the primary RNA transcript. Alternatively, the coding region utilized in the vectors may contain endogenous enhancers/promoters, splice junctions, intervening sequences, polyadenylation signals, etc. or a combination of both endogenous and exogenous control elements.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “sample” refers to any mixture comprising a cell, e.g., tissue. Biological samples may be obtained from animals (including humans) and encompass fluids, solids, tissues, and gases. Biological samples include blood products, such as plasma, serum and the like.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

Also contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus and, more particularly, childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the oesophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

### Spatiotemporal Genomic Profiling of Rare Cancer Cells

Most genomic analyses are done on large, pooled cell populations where unique and rare genomic signatures become diluted among the greater cell population. As such, the majority of these studies cannot resolve the molecular signatures of small cellular sub-populations or rare cells within the larger population.

Furthermore, any spatial information mapping genomic profiles back to specific cells is lost and temporal data describing how a tumor evolves is rarely captured, since most samples are from a single point in time. Even when laser capture microdissection is employed, any real time or dynamic information of cellular behavior, such as invasive potential, proliferation rate, or interactions with the tumor microenvironment, is difficult to capture. A technique that could better connect the dynamic behavior and location of cells with their genomic profiles, could potentially uncover rare molecular profiles that drive tumor progression.

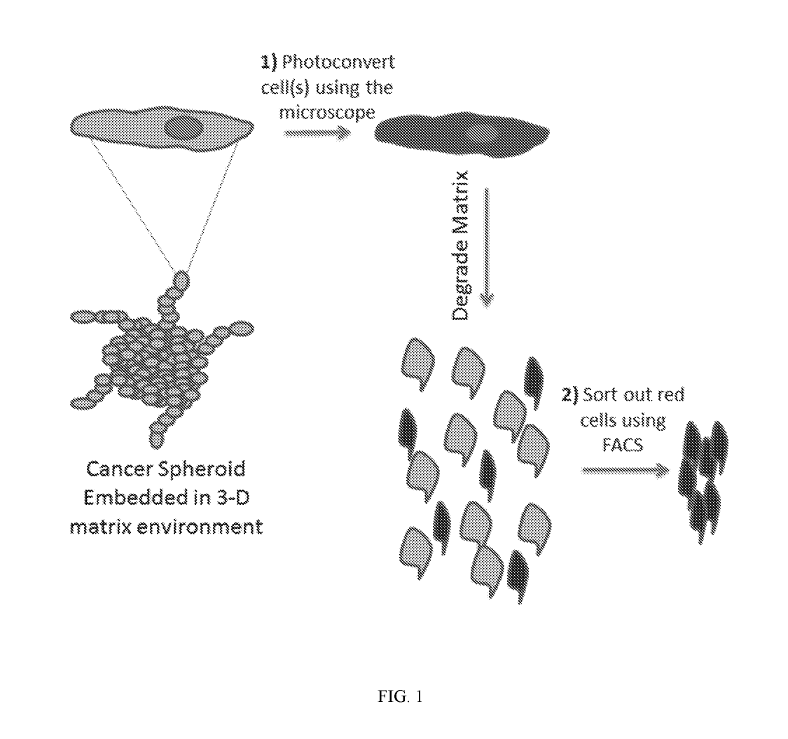

Disclosed herein is a method that can precisely select any living lung cancer cell or group of cells based upon a dynamic phenotype of interest, sort out these cells, and then subject them to genomic analysis. This methodology has been tested in 3-D in vitro for lung cancer spheroids. The technique utilizes a photoconvertible protein, such as Dendra2, which emits green fluorescence similar to GFP, but when excited by 405 nm light, green fluorescence is converted to red fluorescence and cells become photomarked. In this manner, one can optically highlight any living cell of interest with extreme precision, then sort marked cells out from the greater population using fluorescence activated cell sorting (FACS), and finally subject individual cells to genomic analysis (FIG. 1).

The method can be used in laboratories to identify genomic signatures of rare cancer cell populations or single cells of interest. For example, could identify the genomic signature of a single invasive cell, single proliferative cell, single drug-resistance cell, etc.

The method can also be used to create entirely new cell lines derived from any phenotype of interest. For example, new cell lines were created from a highly invasive leader cell, from non-invasive cell, follower cells, etc. Since these are the cells that drive tumor progression, these cell lines could be unique resources for drug screening. The method can be used to identify completely new biomarkers and signatures or used as a clinical diagnostic where single living cells are extracted from live biopsies and subjected to genomic analysis. Treatment can then be guided by this genomic data.

### Photo-Convertible Proteins or Dye for Tracking Cells

A “photo-convertible protein” refers to a polypeptide sequence that changes it molecular structure or three dimensional folding confirmation upon exposure to light or other electromagnetic radiation, e.g. UV or visible light, resulting an altered physical property such as a change in fluorescence. A typical photoconvertible protein is a photoconvertible fluorophore which changes fluorescent properties due to the changes it molecular structure or three dimensional folding confirmation. A photoconvertible fluorophore may display reversible photoactivation or irreversible photoactivation. One example of a photoconvertible fluorescent protein is derived from the Aequorea genus of jellyfish which in unactivated form is non-fluorescent and upon activation emits green light. Enhanced forms of this protein, such as those containing a histidine substitution at the 203 position, have been developed and are reported in the literature, notably by Stepanenko et al., “Fluorescent proteins as biomarkers and biosensors: throwing color lights and molecular and cellular processes,” Curr. Protein Pept. Sci. 9: 338-369 (2008). The histidine-substituted protein, when exposed to intense illumination at 400 nm, displays a hundred-fold increase in absorption at 490 nm and a corresponding increase in fluorescence emission. Other proteins that emit red fluorescence upon exposure to light are Dendra2, IrisFP, tdEosFP, mEos2, PA-Cherryl, mKikGR, Fast-FT, Medium-FT, and Slow-FT. Still further examples are proteins known in the art as Kindling fluorescent proteins, which are photoconvertible at 525-570 nm, and Dronpa proteins, which are photoconvertible at 400 nm. Kindling proteins are described by Chudakov et al., “Chromophore environment provides clue to kindling fluorescent protein riddle,” J. Biol. Chem., 278(9): 7215-7219 (2003), and Dronpa proteins described by Ando et al., “Regulated Fast Nucleocytoplasmic Shuttling Observed by Reversible Protein Highlighting,” Science 306(5700): 1370-1373 (2004).

Another class of photoconvertible proteins are photoswitchable. i.e., which undergo a shift in emission wavelength upon exposure to light. Certain Kindling proteins, described in Chudakov et al., “Photoswitchable cyan fluorescent protein for protein tracking,” Nature Biotechnol. 22: 1435-1439 (2004), are photoswitchable. These proteins have an emission maximum that peaks at 402 nm until irradiated at 405 nm, whereupon the emission maximum shifts to 511 nm. Another example is Kaede, as described in Ando et al., “An optical marker based on the UV-induced green-to-red photoconversion of a fluorescent protein,” Proc. Natl. Acad. Sci. USA 99(20): 12651-12656 (2002). The emission maximum of Kaede shifts from 518 nm to 582 nm upon irradiation at 350-400 nm.

Transformation of a photoconvertible protein can also be achieved by photobleaching, or the conversion of fluorescent proteins that are otherwise display a fluorescent response upon activation by incident light to a protein that is not responsive to the same light. Photobleaching can be permanent or transitory. The light can be at the same wavelength that which is otherwise used to cause the protein to fluoresce.

The transforming light or other electromagnetic radiation can be applied either by successive or single-point exposure or by a patterned simultaneous exposure of all cites to be exposed. Identification of the cells of interest can be performed either before or after the entire population is fluorescent. One means of identification, particularly when the cells are arranged in a fixed array is to place a sample containing cells in the focal plane of a scanning and imaging system that produces a two- or three-dimensional image of the sample and charts the coordinates of the cells of interest in the image. The image can for example be recorded in a charge-coupled device (CCD) and transmitted to a computer system that determines the coordinates of the cells bearing the characteristic of interest. The charted coordinates can then be used to direct light or other electromagnetic radiation to the cells at those coordinates or, when cells other than the cells of interest are to be transformed, the light or other electromagnetic radiation can be directed to locations other than those of the charted coordinates. In either case, the light or other electromagnetic radiation is applied in an area-patterned manner, i.e., in a pattern coincident with the fixed locations of the cells of interest. In the case of cell growth for dividing cells the light or other electromagnetic radiation may be directed to an area occupied by the newly formed cells.

Once the cells to be transformed are identified, the patterned exposure of the cells can be achieved either in a single-point successive manner (one cell at a time or one well at a time of a multi-well array where cells reside in each well) or all at once, or a combination in which segments of the area occupied by the cells are exposed in succession.

In certain embodiments, for any of the methods disclosed herein that utilize a photo-convertible protein, it is contemplated that a photo-convertible dye may also be used, such as, cyanine-based dyes 3.5 or 5.5, N-methyl-diazaxanthilidene, 1,1′,3,3,3′,3′-hexamethylindotrycarbocyanine iodide (HITC), 1,1′-dioctadecyl-3,3,3′,3′-tetramethylindotricarbocyanine iodide. Carlson et al. report a photoconversion technique to track individual cells in vivo using a commercial lipophilic membrane dye that exhibits a permanent fluorescence emission shift (photoconversion) after light exposure. PLoS One. 2013; 8(8): e69257. Also contemplated is SYTO62.

### Genetic Profiling

After extraction and isolation of nucleic acids from cells the sequences may be determined. Often, a sequencing method is classic Sanger sequencing. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS)(Helicos), massively-parallel sequencing, Clonal Single Molecule Array (Solexa), shotgun sequencing, Maxim-Gilbert sequencing, primer walking, sequencing using PacBio, SOLiD, Ion Torrent, or Nanopore platforms and any other sequencing methods known in the art.

In some examples, sequencing can be performed from samples that may comprise a variety of different types of nucleic acids. Nucleic acids may be polynucleotides or oligonucleotides. Nucleic acids included, but are not limited to DNA or RNA, single stranded or double stranded or a RNA/cDNA pair.

Early detection and monitoring of genetic diseases, such as cancer is often useful and needed in the successful treatment or management of the disease. One approach may include the monitoring of a sample derived from rare cells with a population of polynucleotides. In some cases, disease may be characterized or detected based on detection of genetic aberrations, such as a change in copy number variation and/or sequence variation of one or more nucleic acid sequences, or the development of other certain genetic alterations

Generally, the methods comprise sample preparation, or the extraction and isolation of nucleic acid sequences from a cells; subsequent sequencing of the nucleic acids by techniques known in the art; and application of bioinformatics tools to detect mutations and copy number variations as compared to a reference. The methods also may contain a database or collection of different rare mutations or copy number variation profiles of different diseases, to be used as additional references in aiding detection of mutations, copy number variation profiling or general genetic profiling of a disease.

In some embodiments, the methods of the disclosure may comprise selectively enriching regions from the genome or transcriptome of a cell prior to sequencing. In certain embodiments, methods of the disclosure comprise attaching one or more barcodes to the nucleic acids or fragments thereof prior to any amplification or enrichment step. In some embodiments, amplification comprises selective amplification, non-selective amplification, suppression amplification or subtractive enrichment.

In some embodiments, a genetic variant, mutation or copy number variation occurs in a region of the genome selected from the group consisting of gene fusions, gene duplications, gene deletions, gene translocations, microsatellite regions, gene fragments or combination thereof. In other embodiments a genetic variant, mutation, or copy number variation occurs in a region of the genome selected from the group consisting of genes, oncogenes, tumor suppressor genes, promoters, regulatory sequence elements, or combination thereof. In some embodiments the variant is a nucleotide variant, single base substitution, or small indel, transversion, translocation, inversion, deletion, truncation or gene truncation.

In some embodiments, samples at succeeding time intervals from the same cell are analyzed and compared to previous sample results. The method of the disclosure may further comprise determining partial copy number variation frequency, loss of heterozygosity, gene expression analysis, epigenetic analysis and hypermethylation analysis.

In some embodiments, the methods of the disclosure comprise normalizing and detection is performed using one or more of hidden markov, dynamic programming, support vector machine, Bayesian network, trellis decoding, Viterbi decoding, expectation maximization, Kalman filtering, or neural network methodologies.

In some embodiments the methods of the disclosure comprise monitoring disease progression, monitoring residual disease, monitoring therapy, diagnosing a condition, prognosing a condition, or selecting a therapy based on discovered variants.

In some embodiments, a therapy is modified based on the most recent sample analysis. Further, the methods of the disclosure comprise inferring the genetic profile of a tumor, infection or other tissue abnormality. In some embodiments, growth, remission or evolution of a tumor, infection or other tissue abnormality is monitored. In some embodiments the subject's immune system are analyzed and monitored at single instances or over time.

In some embodiments, the methods of the disclosure comprise identification of a variant that is followed up through an imaging test (e.g., CT, PET-CT, MRI, X-ray, ultrasound) for localization of the tissue abnormality suspected of causing the identified variant.

In the early detection of cancers, any of the methods herein described, including mutation detection or copy number variation detection may be utilized to detect cancers. These system and methods may be used to detect any number of genetic aberrations that may cause or result from cancers.

Additionally, the methods described herein may also be used to help characterize certain cancers. Genetic data produced from the system and methods of this disclosure may allow practitioners to help better characterize a specific form of cancer. Often times, cancers are heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer.

The methods provided herein may be used to monitor cancers, or other diseases in a particular subject. This may allow either a subject or practitioner to adapt treatment options in accord with the progress of the disease. In this example, the methods described herein may be used to construct genetic profiles of a particular subject of the course of the disease. In some instances, cancers can progress, becoming more aggressive and genetically unstable. In other examples, cancers may remain benign, inactive, dormant or in remission. The system and methods of this disclosure may be useful in determining disease progression, remission or recurrence.

Further, the methods described herein may be useful in determining the efficacy of a particular treatment option. In one example, successful treatment options may actually increase the amount of copy number variation or mutations if the treatment is successful. In other examples, this may not occur. In another example, perhaps certain treatment options may be correlated with genetic profiles of cancers over time. This correlation may be useful in selecting a therapy. Additionally, if a cancer is observed to be in remission after treatment, the methods described herein may be useful in monitoring residual disease or recurrence of disease.

For example, mutations occurring within a range of frequency beginning at threshold level can be determined from DNA in a sample from a subject, e.g., a patient. The mutations can be, e.g., cancer related mutations. The frequency can range from, for example, at least 0.1%, at least 1%, or at least 5% to 100%. The sample can be a tumor sample of cell free DNA. A course of treatment can be prescribed based on any or all of mutations occurring within the frequency range including, e.g., their frequencies. A sample can be taken from the subject at any subsequent time. Mutations occurring within the original range of frequency or a different range of frequency can be determined. The course of treatment can be adjusted based on the subsequent measurements.

The methods described herein are not be limited to detection of mutations and copy number variations associated with only cancers. Various other diseases and infections may result in other types of conditions that may be suitable for early detection and monitoring. For example, in certain cases, genetic disorders or infectious diseases may cause a certain genetic mosaicism within a subject. This genetic mosaicism may cause copy number variation and mutations that could be observed. In another example, the system and methods of the disclosure may also be used to monitor the genomes of immune cells within the body. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing.

Further, the methods of this disclosure may also be used to monitor systemic infections themselves, as may be caused by a pathogen such as a bacteria or virus. Copy number variation or even rare mutation detection may be used to determine how a population of pathogens are changing during the course of infection. This may be particularly important during chronic infections, such as HIV/AIDs or Hepatitis infections, whereby viruses may change life cycle state and/or mutate into more virulent forms during the course of infection.

Early detection and monitoring of genetic diseases, such as cancer is often useful and needed in the successful treatment or management of the disease. Cell free DNA (“cfDNA”) may contain genetic aberrations associated with a particular disease. One approach may include the monitoring of a sample derived from cell free nucleic acids that can be found in different types of bodily fluids, e.g. blood, urine, saliva, etc. In some cases, disease may be characterized or detected based on detection of genetic aberrations, such as a change in copy number variation and/or sequence variation of one or more nucleic acid sequences, or the development of other certain rare genetic alterations.

### EXAMPLES

### Spatiotemporal Genomic and Cellular Analysis (SaGA)

SaGA is a method where one can image live cells, pick any cell or group of cells wanted from a biologically relevant 3-D environment, extract the cell(s), and subject them to genomic analysis. SaGA is used to precisely select living cells based upon their behavior (phenotype) and subject them to genomic analysis.

The steps to this methodology include

1) Photoconversion to Select Cancer Cells

Dendra2 is a photoconvertible fluorophore which emits green fluorescence similar to GFP. However, when excited by 405 nm light, green fluorescence is converted to red fluorescence due to cleavage of histidine 62, an event termed photoconversion. Therefore, single cell precision, any cancer cell expressing Dendra2 can be optically highlighted (turned red) using a standard point scanning confocal microscope. A region of interest is drawn around the cell(s) of interest, based upon any phenotype visible by transmitted light or fluorescent protein tags. The software uses this region to guide a ˜3-5 sec excitation with the 405 nm laser, resulting in near instantaneous photoconversion of Dendra2, and photomarking the cell red. Using this approach, we can photoconvert about 50-100 individual cells in 1-2 hr. Single cells can be photoconverted, without inducing any measurable photoconversion of neighboring cells.

2) Cell Extraction and Sorting

Cells are extracted from the 3-D environment using dispase for 15 min. (collagenase for collagen). Cells are then sorted to separate red photoconverted cells from green cells using a standard cell sorter. A BD FACS Aria II cell sorter was use the which is capable of sorting 30-50 red photoconverted cells from a population of 5-10,000 green cells.

3) Cell Line Creation and Genomic Analysis

Once the cells are sorted, one can grow them in culture using standard cell culture techniques. Purified subcultures have been living for over 1.5 years and display the same initial phenotype. In this way, it appears they can be amplified it to virtually unlimited quantities allowing one to perform genomic, epigenomic, and proteomic profiling of rare cell types.

SaGA uses fluorescence imaging to isolate user-defined cells (as opposed to random selection) from a biologically relevant environment, then extract and amplify these cells with cell sorting. In this manner, one can use a phenotype or behavior of interest (e.g., rare, highly invasive, highly proliferative) to decide which rare cells to sequence, and thus extract and identify unique mutations in rare cells that are driving cancer cell populations. In the method one selects living cells in space and over time and subject them to genomic analysis or other experimental approaches.

Unique mutation profiles were found in leader and follower cells that are not currently part of any diagnostic or therapeutic approaches. These mutations are now ready to be validated in cell lines and probed in lung cancer patients. If these mutations in rare cells are common among patients, then large-scale sequencing efforts (e.g., TCGA) which grind up entire tumor tissues, are missing a hidden and rare mutation profile that drives the tumor. The consequences of finding these rare mutations are far-reaching and would impact cancer therapeutics and diagnostics by allowing us to create the first rare cell genomic panel that is integrated into clinical care.

SaGA can be performed directly on clinical samples to discover mutations directly in patient samples, determine how rare cells respond to treatments, perform drug screens on rare cells, and provide treatment for actionable rare cell mutations.

1. A method of selecting unique cancer cells comprising:
a) mixing a group of cells suspected of containing cancer cells with a fluorescent photo-convertible protein, dye, or a recombinant vector configured to expresses a fluorescent photo-convertible protein, wherein the fluorescent photo-convertible protein or dye is configured to change fluorescent emissions if exposed to a predetermined wavelength of electromagnetic radiation, under conditions such that the group of cells contain the fluorescent photo-convertible protein or dye providing fluorescent protein or dye containing cells;
b) providing conditions such that the fluorescent protein or dye containing cells replicate providing fluorescent replicated cells;
c) identifying a fluorescent replicated cell that is expressing a physical characteristic that is unique compared to the other fluorescent replicated cells providing a unique fluorescent replicated cell;
d) exposing the unique fluorescent replicated cell to the predetermined wavelength of electromagnetic radiation and not exposing the predetermined wavelength to the other fluorescent replicated cells under conditions such that the unique fluorescent replicated cell changes fluorescent emissions providing a changed unique fluorescent replicated cell; or
exposing the other fluorescent replicated cells to the predetermined wavelength of electromagnetic radiation and not exposing the predetermined wavelength to the unique fluorescent replicated cells under conditions such that the other fluorescent replicated cells change fluorescent emissions providing changed other fluorescent replicated cells;
and
e) separating the changed unique fluorescent replicated cell from the other fluorescent replicated cells or separating the unique fluorescent replicated cells from the changed other fluorescent replicated cells.

2. The method of claim 1, further comprising the step of replicating the changed unique fluorescent replicated cell or the unique fluorescent replicated cells.

3. The method of claim 1, wherein the physical characteristic that is unique is a location of the fluorescent replicated cell that are outward from a central mass of fluorescent replicated cells.

4. The method of claim 1, wherein the physical characteristic that is unique is a location of the fluorescent replicated cell in a shape that is distinct from the shape of a central mass of fluorescent replicated cells.

5. The method of claim 1, wherein the physical characteristic that is unique is a location of the fluorescent replicated cell at the tip of an arm of fluorescent replicated cells.

6. The method of claim 1, wherein the mixture of cells is a tumor, organ biopsy, blood cells, urine cells, skin cells, tongue cells, cheek cells, fecal cells, or vaginal cells.

7. The method of claim 1, wherein the mixture of cells are a lung cells, brain cells, kidney cells, pancreatic cells, ovarian cells, prostate cells or tumors thereof.

8. The method of claim 1, wherein the unique fluorescent replicated cell is an invasive cancer cell.

9. The method of claim 1, wherein the fluorescent photo-convertible protein is selected from Kaede, KikGR, EosFP, and Dendra2.

10. The method of claim 1, further comprising extracting nucleic acids from the unique fluorescent replicated cell providing extracted nucleic acids.

11. The method of claim 10, further comprising sequencing the extracted nucleic acids providing a unique florescent replicated cell sequence.

12. The method of claim 11, further comprising comparing the unique florescent replicated cell sequence to a standard or normal sequence and identifying similarities or differences between unique florescent replicated cell sequence and the standard or normal sequence.

13. The method of claim 12, further comprising recording the similarities or differences on a computer readable medium.

14. A composition comprising cells made by the process of claim 1.
